(19) United States
(12) Patent Application Publication
(10) Pub. No.: US 2010/0239510 A1
(43) Pub. Date: Sep. 23, 2010

(54) SKIN-CARE COMPOSITION COMPRISING DILL EXTRACT

(76) Inventors: Robert Bao Kim Ha, Liberty, OH (US); Rosemarie Osborne, Oxford, OH (US); Richard John Szczepaniak, Liberty, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/691,471

(22) Filed: Jan. 21, 2010

Related U.S. Application Data
(60) Provisional application No. 61/146,543, filed on Jan. 22, 2009.

(51) Int. Cl. A61K 8/97; A61K 36/23; A61Q 17/04

(52) U.S. Cl. 424/59; 424/725

(57) ABSTRACT

A skin-care composition may comprise a dill extract, a safe and effective amount of a peptide, a safe and effective amount of a hydrating skin active, and a dermatologically acceptable carrier. The skin-care composition may further comprise a safe and effective amount of an acute skin-care active. Further, a method of producing a chronic skin-firming benefit and, optionally, an acute skin-tightening sensation, said method comprising the step of topically applying to the skin of a mammal in need of such treatment, a safe and effective amount of the skin-care composition.

### CROSS REFERENCE TO RELATED APPLICATIONS

This application claims the benefit of Provisional Application Ser. No. 61/146,543, filed Jan. 22, 2009.

### FIELD OF THE INVENTION

The present invention relates to a skin-care composition comprising dill extract to improve skin elasticity. Using a combination of skin-care actives, the skin-care composition also improves skin hydration and skin condition.

### BACKGROUND OF THE INVENTION

Currently, a number of skin-care products are available to consumers which claim to improve the health and physical appearance of keratinous tissues such as the skin, hair, and nails. There is a need for a multi-functional skin-care active combination that delights consumers with its skin firming, hydrating, and conditioning benefits. Specifically, there is a need for a skin-care composition that remedies the laxity, slackness, or the like condition of skin that occurs as a result of loss of, damage to, alterations to, and/or abnormalities in dermal elastin. Without being limited by theory, it has been found that dill extract can both prevent development of sagging and reverse existing sagging of the skin. It has also been found that dill extract compositions stabilize cellular and extracellular components of keratinous tissue, particularly the proteinaceous components such as elastin and collagen and the cell membranes of such tissue. The dill extract treated proteins and cells become stronger, more resistant, and are able to withstand higher levels of the above-described insults. There is also a need for skin-care formulations that provide a “signal” to the consumer that the product is working.

### SUMMARY OF THE INVENTION

In one embodiment, a skin-care composition comprises a dill extract, a safe and effective amount of a peptide, a safe and effective amount of a hydrating skin active, and a dermatologically acceptable carrier.

In another embodiment, a skin-care composition comprises a dill extract, a safe and effective amount of a peptide, a safe and effective amount of a hydrating skin active, a safe and effective amount of an acute skin-care active, and a dermatologically acceptable carrier.

In yet another embodiment, a skin-care composition comprises a dill extract; a safe and effective amount of a peptide selected from the group consisting of palmitoyl pentapeptide-3, carnosine, palmitoyl dipeptide-7, and combinations thereof; a safe and effective amount of a hydrating skin active selected from the group consisting of niacinamide, caffeine, and combinations thereof; a safe and effective amount of an acute skin-care active selected from the group consisting of polyethylene, polyimide-1, and combinations thereof; and a dermatologically acceptable carrier.

### DETAILED DESCRIPTION OF THE INVENTION

While the specification concludes with the claims particularly pointing out and distinctly claiming the invention, it is believed that the present invention will be better understood from the following description.

All ratios are weight ratios, unless specifically stated otherwise. All ranges are inclusive and combinable; therefore, every range given throughout this specification will include every narrower range that falls within such broader range as if such narrower ranges were all expressly written herein. The number of significant digits conveys neither a limitation on the indicated amounts nor on the accuracy of the measurements. All measurements are understood to be made at ambient conditions. All such weights as they pertain to listed ingredients are based on the active level and do not include carriers or by-products that may be included in commercially available materials, unless otherwise specified.

As used herein, “comprising” means that other steps and ingredients can be added. This term encompasses the terms “consisting of” and “consisting essentially of” The phrase “consisting essentially of” means that the composition may include additional ingredients, but only if the additional ingredients do not materially alter the basic and novel characteristics of the claimed compositions or methods.

The term “skin-care composition,” as used herein, means a composition that may be applied to mammalian keratinous tissue without undue undesirable side effects.

As used herein, “dill extract” refers to dill, dill seed, dilly, peucedanum graveolens, anethum graveolens, anethum sowa, and related synthetic and derivative materials.

The terms “skin-care actives” and “skin benefit agents,” as used herein, mean compounds that, when applied to the skin, provide an acute and/or chronic benefit to keratinous tissue, including the skin.

The term “keratinous tissue,” as used herein, refers to keratin-containing layers disposed as the outermost protective covering of mammals and includes, but is not limited to, skin, hair, nails, etc.

The terms “elastin” and “dermal elastin,” as used herein, refer to a protein in keratinous tissue that helps control skin elasticity.

The term “topical application,” as used herein, means to apply or spread the compositions of the present invention onto the surface of the keratinous tissue.

The term “dermatologically acceptable,” as used herein, means that the compositions or components thereof so described are suitable for use in contact with human keratinous tissue without undue toxicity, incompatibility, instability, allergic response, and the like.

The term “safe and effective amount,” as used herein, means an amount of a compound or composition sufficient to significantly induce a positive benefit, preferably a positive keratinous tissue appearance or feel benefit, including independently or in combinations the benefits disclosed herein, but low enough to avoid serious side effects, i.e., to provide a reasonable benefit to risk ratio, within the scope of sound judgment of the skilled artisan.

“Benefit,” as used herein in reference to mammalian skin, means an acute or chronic benefit to the skin. Benefits may regulate and/or improve skin. Some non-limiting examples of benefits include improving skin elasticity; improving skin hydration; and improving skin condition.

To “improve skin elasticity,” “improve firmness,” or “increase firmness,” as used herein, means preventing or delaying loss of skin or hair elasticity (e.g., due to loss, damage and/or inactivation of functional skin elastin, resulting in such conditions as elastosis, sagging, loss of skin or hair recoil from deformation).

The term “sagging” as used herein means the laxity, slackness, or the like condition of skin that occurs as a result of loss of, damage to, alterations to, and/or abnormalities in dermal elastin.

To “improve skin hydration,” as used herein, means increasing skin moisturization or alleviating chronic and/or acute dry skin.

To “regulate skin condition” means maintaining skin appearance and/or feel with little to no degradation in appearance and/or feel.

To “improve skin condition,” as used herein, means affecting a positive change in skin appearance and/or feel. The skin appearance and/or feel benefit may be a chronic benefit and may include one or more of the following: thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); reducing cellulite; reduce the breakdown of collagen; reducing the appearance of discoloration to the skin, for example, under-eye circles, blotchiness (e.g., uneven red coloration due to, for example, rosacea), sallowness (pale or yellow color); reducing puffiness and/or swelling; reducing the appearance of hyperpigmented skin regions such as age spots and freckles; thickening the skin to reduce atrophy (e.g., of the skin), increasing the convolution of the dermal-epidermal border, non-melanin skin discoloration such as under eye circles, discoloration caused by telangiectasia or spider vessels, discolorations due to melanin (e.g., pigment spots, age spots, uneven pigmentation) and other chromophores in the skin (e.g., lipofuscin, protein crosslinks such as those that occur with glycation, and the like); delaying the onset and/or reducing the appearance of fine lines, wrinkles, frown lines, expression lines, rhytides, blemishes, photodamage, crevices, and/or unevenness on the skin; and other histological changes in the stratum corneum, dermis, epidermis, vascular system (e.g., telangiectasia or spider vessels), and underlying tissues (e.g., fat and/or muscle), especially those proximate to the skin.

The skin-care composition of the present invention comprises a dermatologically-acceptable carrier, a combination of chronic skin-care actives, and optionally an acute skin-care active. The composition may contain other ingredients, such as thickening agents, structuring agents, silicone elastomers, and mixtures thereof in order to modify the viscosity and/or feel of the composition.

The composition has a viscosity of from about 1,000 centipoise (cps) to about 200,000 cps, more preferably from about 10,000 cps to about 100,000 cps, and most preferably about 20,000 cps to about 60,000 cps. Viscosities are measured on a Brookfield viscometer using a T-C bar spindle with a heliopath setting at 5 rpm at 25° C.

The compositions of the present invention, including the essential and optional components thereof, are described in detail hereinafter.

### I. Carrier

The skin-care composition of the present invention may comprise from about 10% to about 99.9% of a dermatologically acceptable carrier. The characteristics of the carrier utilized in the present invention depend on the type of product form desired for the composition. The topical compositions useful in the subject invention may be made into a wide variety of product forms such as are known in the art. These include, but are not limited to, lotions, creams, sticks, sprays, ointments, pastes, mousses and cosmetics.

In one embodiment, the carrier is in the form of an aqueous gel. Gel systems may comprise a gelling agent and a solvent. Typically, for aqueous gels, water is a suitable solvent; however, water may be substituted by or supplemented with other aqueous solvents such as polyols (e.g., glycerin, butylene glycol, pentylene glycol, etc.). The aqueous gel may comprise about 60% to about 95% of the aqueous solvent. Suitable gelling agents generally may include water-soluble or colloidally water-soluble polymers, which includes synthetic and natural polymers, and particulate-based materials. Other suitable gelling agents may comprise carboxylic, natural, particulate, acrylate or acrylamide copolymers and their salts. In a preferred embodiment, the carrier is a polymeric thickener that can be anionic, cationic, or nonionic. Typically, the skin-care composition may comprise from about 0.05% to about 3% of the gelling agent.

In other embodiments, the carrier of the present invention is in the form of an emulsion. Herein, “emulsions” generally contain an aqueous phase and an oil phase. The oils may be derived from animals, plants, or petroleum, may be natural or synthetic, and may include silicone oils. Emulsion carriers include, but are not limited to, oil-in-water, water-in-oil, silicone-in-water, water-in-silicone, water-in-oil-in-water, and oil-in-water-in-silicone emulsions. In a particular embodiment, the dermatologically acceptable carrier comprises an oil-in-water emulsion, resulting in an opaque, lotion-like composition. In another embodiment, the carrier comprises a silicone-in-water emulsion having a very small silicone phase—for instance, 1-2% silicone. The emulsion further may comprise a humectant, for example, glycerin and a non-ionic, cationic and/or anionic emulsifier. Suitable emulsifiers are disclosed in, for example, U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986).

### II. Chronic Actives

The skin-care composition may comprise a combination of chronic skin actives which, over a period of time, improve skin firmness, hydration, and condition. To meet these goals, the compositions of the present invention comprise A. dill extract, B. peptides, and C. vitamin B₃, caffeine, or both.

A. Firming

The composition of the present invention may comprise one or more types of dill extract to improve skin elasticity and increase skin firmness. Dill extract restores elastin functionality and encourages LOXL (the major component in skin elasticity during aging) synthesis. Restoration of LOXL levels in aged skin can restore the skin's elasticity and firmness and prevent sagging. An aqueous extract of dill has been found to be an active, dose-responsive and reproducible material, producing a 2-fold increase in LOXL gene expression (RT-PCR) and 1.5-fold increase in elastin gene expression. In a proprietary 3D skin model (Mimeskin), 0.0750% dill extract produced a significant (p<0.001) increase in LOXL mRNA (64%) and LOXL protein (12%) at four weeks, producing microfilaments containing elastin as detected by transmission electron microscopy. To determine if the microfilaments were functional, clinical instrumentation (tribometer) was used to evaluate the skin model. Dill extract (0.0750%) produced a significant (p<0.01) 29% increase in elasticity versus a control after four weeks.

The skin-care composition may comprise about 0.001% to about 7.0% dill extract, more preferably from about 0.01-5.0%, and most preferably from about 0.05-2.5%. In a preferred embodiment, the dill extract is present as peucedanum graveolens in LYS'LASTINE V, commercially available from BASF.

B. Hydrating

The composition of the present invention may comprise one or more chronic skin actives to increase skin hydration. In other embodiments, the chronic skin active may transform glycerol throughout skin to provide a hydrating benefit. The hydrating skin active of the present invention may comprise caffeine, one or more vitamin B₃ compounds, or a combination thereof. When present, the composition preferably includes from about 0.01% to about 10%, more preferably from about 0.1% to about 5%, by weight of the composition, of caffeine.

Non-limiting examples of vitamin B₃ useful herein include niacinamide, tocopherol nicotinate, and mixtures thereof. As used herein, “vitamin B₃ compound” means a compound having the formula:

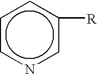

wherein R is —CONH₂ (i.e., niacinamide), —COOH (i.e., nicotinic acid) or —CH₂OH (i.e., nicotinyl alcohol); derivatives thereof; and salts of any of the foregoing. When present, the composition preferably includes from about 0.01% to about 50%, more preferably from about 0.1% to about 10%, by weight of the composition, of vitamin B₃. In a preferred embodiment, vitamin B₃ is niacinamide.

C. Conditioning

The composition of the present invention may comprise one or more peptides to improve skin condition. In some embodiments, the peptide may improve skin appearance and decrease the breakdown of collagen. As used herein, “peptide” refers to peptides containing ten or fewer amino acids, their derivatives, isomers, and complexes with other species such as metal ions (e.g., copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, and Cu-histidine-glycine-glycine (Cu-HGG, also known as IAMIN®). A further example includes carnosine (beta-alanine-histidine). Preferred peptides include PROMATRIXYL, comprising palmitoyl pentapeptide-3 and PALESTRINA, comprising palmitoyl dipeptide-7, both available from Croda Inc.

The composition may comprise from about 1×10⁻⁷% to about 20%, alternatively from about 1×10⁻⁶% to about 10%, and alternatively from about 1×10⁻⁵% to about 5% of a peptide. In a preferred embodiment, the composition may comprise from about 0.1% to about 2% of a peptide.

This multi-functional skin-care active combination comprising dill extract, a peptide, and caffeine and/or vitamin B₃ delights consumers with its skin firming, hydrating, and conditioning benefits.

### III. Acute Actives

Consumers are typically reluctant to use a product that does not provide a short-term, recognizable benefit. Accordingly, in some embodiments of the present invention, in addition to the long term or chronic benefits provided by the above-referenced skin-care actives, it may also be desirable to include an acute skin-care active. Skin sensations caused by the application of skin-care products, such as a localized and instantaneous tightening feel to the skin, warmth to the skin, and/or skin tingling are consumer preferable signals/indications that the skin-care product is working.

In one embodiment, the acute skin-care active incorporates a cooling sensation. Examples of cooling sensates are menthols and menthyl lactate. In a preferred embodiment, the acute skin-care active incorporates a skin-tightening polymer technology to deliver an immediate tightening sensation as the polymer sets up on the skin. By including both chronic skin-care actives and at least one acute skin-care active, the composition is designed to both maximize immediate consumer in-use tightening and deliver clinically effective ingredients to improve skin's structure. The acute tightening incentivizes consumers to continue using the product to reap the long-term benefits from the chronic skin-care actives. Examples of acute skin-care actives are polyvinylpyrrolidone and hydrolyzed wheat protein. A preferred acute skin-care active is a mixture of polyimide-1 and water, available under the tradename Aquaflex XL-30 from International Specialty Products (ISP) Inc. In the composition of the present invention, the acute skin-care active preferably comprises from about 0.0001-25% by weight of the composition, more preferably from about 0.01-10%, and most preferably from about 0.1-5%.

### IV. Optional Actives

The skin-care composition may further comprise one or more optional skin-care actives to provide benefits in addition to the benefits already mentioned. It should be noted, however, that many skin-care actives may provide more than one benefit, or operate via more than one mode of action. Therefore, classifications herein are made for the sake of convenience and are not intended to limit the active to that particular application or applications listed.

A. Vitamins

The composition of the present invention may comprise one or more vitamins, for example, to provide antioxidant and/or other nutritional benefits to the skin. Herein, “vitamins” means vitamins, pro-vitamins, and their salts, isomers and derivatives. The vitamins may include water soluble vitamins, for example, nicotinic acid, C1-C18 nicotinic acid esters, and nicotinyl alcohol; B6 compounds, such as pyroxidine; and B5 compounds, such as panthenol, or “pro-B5”); and Vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as Vitamin A compounds, and all natural and/or synthetic analogs of Vitamin A, including retinoids, carotenoids, and other compounds which possess the biological activity of Vitamin A; Vitamin D compounds; Vitamin E compounds, or tocopherol, including tocopherol sorbate, tocopherol acetate, other esters of tocopherol; Vitamin K compounds; and mixtures thereof. In one embodiment, the compositions of the instant invention may comprise from about 0.0001% to about 10%, alternatively from about 0.001% to about 8%, alternatively from about 0.01% to about 5%, and alternatively from about 0.1% to about 1%, of the vitamin.

B. Sugar Amines

The composition of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Examples of sugar amines useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine. Additionally, combinations of two or more sugar amines may be used. In one embodiment, the composition comprises from about 0.01% to about 15%, alternatively from about 0.1% to about 10%, and alternatively from about 0.5% to about 5%, of the sugar amine.

C. Sunscreens

The compositions of the subject invention may comprise one or more sunscreen actives (or sunscreen agents). Herein, suitable sunscreen actives include oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof.

D. Protease Inhibitors

The composition of the present invention may comprise protease inhibitors. Suitable protease inhibitors include, but are not limited to, hexamidine, vanillin acetate, menthyl anthranilate, and mixtures thereof. The skin-care compositions of the present invention may comprise a safe and effective amount of one or more hexamidine compounds, its salts, isomers, tautomers, and derivatives. As used herein, “hexamidine” means a compound having the formula:

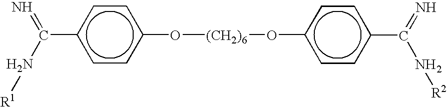

wherein R¹ and R² are optional or are organic acids (e.g., sulfonic acids, etc.). Preferably, the hexamidine is hexamidine isethionate, available under the tradename Elastab® HP100 from Laboratoires Serobiologiques (Pulnoy, France). In the composition of the present invention, the hexamidine preferably comprises from about 0.0001-25% by weight of the composition, more preferably from about 0.001-10%, more preferably from about 0.01-5%, and most preferably from about 0.02-2.5%.

E. Other Skin-Care Actives

The composition of the present invention may comprise non-vitamin antioxidants and radical scavengers, preservatives, minerals, oil control agents, flavonoids, phytosterols and/or plant hormones, protease inhibitors, tyrosinase inhibitors, tanning actives, anti-acne actives, desquamation actives, chelating agents, skin lightening agents, hair growth regulators, anti-wrinkle actives, anti-atrophy actives, anti-cellulite agents, anti-inflammatory agents, N-acyl amino acid compounds, antimicrobials, and antifungals. These skin-care actives are provided in further detail in U.S. Patent Application Publication Nos. US 2006/0275237A1 and US 2004/0175347A1. Suitable non-vitamin antioxidants include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), ubiquinone (co-enzyme Q10), tea extracts, including green tea extract, and combinations thereof. Suitable examples of plant sterols and/or plant hormones include, but are not limited to, sitosterol, stigmasterol, campesterol, brassicasterol, kinetin, zeatin, and mixtures thereof. Suitable anti-inflammatory agents include, but are not limited to, glycyrrhizic acid (also known as glycyrrhizin, glycyrrhixinic acid, and glycyrrhetinic acid glycoside), glycyrrhetenic acid, and combinations thereof. Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France). Suitable anti-cellulite agents include, but are not limited to, xanthine compounds (e.g., theophylline, theobromine, and aminophylline).

Other useful skin-care actives include dehydroepiandrosterone (DHEA), its analogs and derivatives; alpha- and beta-hydroxyacids, including glycolic acid and octanoyl salicylate, plant extracts such as cucumber and witch hazel extract, arbutin, dimethyl aminoethanol (DMAE), kojic acid, and dihydroxy acetone (DHA).

### V. Method of Use

The skin-care composition of the present invention may be applied to the skin at an amount and frequency to improve skin elasticity, improve hydration, regulate or improve skin condition, maintain or improve the signs of skin aging, or maintain or improve insult-affected keratinous tissue. In a preferred embodiment, the skin-care composition is used as a specialized treatment for an entire face, with concentrated usage in areas with expression lines. Further, the skin-care composition may be used to produce a chronic skin-firming benefit and, optionally, an acute skin-tightening sensation by topically applying to the skin of a mammal in need of such treatment a safe and effective amount of the skin-care composition.

### VI. Examples

The examples of the present invention may all be prepared by conventional means. The following general making instructions apply to all of the examples provided below: Phase A, B and C materials are prepared in separate containers. Phase A materials are heated to 25-30° C. and blended together. Both Phase B and C materials are mixed at room temperature until uniform. When all phases are ready, Phases B and C are added to Phase A one at a time; mixing Phases A/B/C is continued until uniform. Then add the rest of materials one at a time and continue mixing the batch until it is smooth.

| | Example | | | | | |
|---|---|---|---|---|---|---|
| | 1 | 2 | 3 | 4 | 5 | 6 |
| Phase A | | | | | | |
| Water | QS | QS | QS | QS | QS | QS |
| Glycerin | 2.00 | 2.00 | 2.00 | 2.00 | 2.00 | 2.00 |
| Niacinamide | 5.00 | 5.00 | 0.75 | 7.5000 | 3.5000 | 2.0000 |
| Caffeine | 1.50 | 1.50 | 1.50 | — | 1.50 | 1.50 |
| Lys'Lastine V*¹⁴ | 0.0750 | 1.00 | 3.00 | 5.0000 | 5.00 | 7.50 |
| Nipagin M*¹ | 0.0700 | 0.0700 | 0.0700 | 0.0700 | 0.0700 | 0.0700 |
| Promatrixyl*² | 0.3530 | 0.3530 | 0.3530 | — | 0.3530 | 0.3530 |
| Glycasil-L*³ | 0.1000 | 0.1000 | 0.1000 | 0.1000 | 0.1000 | 0.1000 |
| Benzyl Alcohol | 0.2500 | 0.2500 | 0.2500 | 0.2500 | 0.2500 | 0.2500 |
| Disodium EDTA | 0.1000 | 0.1000 | 0.1000 | 0.1000 | 0.1000 | 0.1000 |
| Palestrina*⁷ | 0.5760 | 0.5760 | 0.5760 | 0.5760 | — | 0.5760 |
| Dexpanthenol*⁸ | 0.5000 | 0.5000 | 0.5000 | 0.5000 | 0.5000 | 0.5000 |
| Hydrolite-5*¹³ | 2.0000 | 2.0000 | 2.0000 | 2.0000 | 2.0000 | 2.0000 |
| Sepiwhite*¹¹ | — | — | 1.0000 | 1.0000 | — | — |
| Hexamidine Isethionate | — | — | — | 0.1000 | — | — |
| N-Acetyl Glucosamine | — | — | 4.0000 | — | — | 2.0000 |
| Phase B | | | | | | |
| Water | 17.0000 | 17.0000 | 17.0000 | 17.0000 | 17.0000 | 17.0000 |
| Ultrez 20*⁶ | 0.2500 | 0.2500 | 0.2500 | 0.2500 | 0.2500 | 0.2500 |
| Phase C | | | | | | |
| Tocopherol Acetate | 0.5000 | 0.5000 | 0.5000 | 0.5000 | 0.5000 | 0.5000 |
| Brij 30*⁵ | 0.1000 | 0.1000 | 0.1000 | 0.1000 | 0.1000 | 0.1000 |
| Microthine 510*¹⁰ | 1.0000 | 1.0000 | 1.0000 | 1.0000 | 1.0000 | 1.0000 |
| Dow Corning 1503*¹² | 1.0000 | 1.0000 | 1.0000 | 1.0000 | 1.0000 | 1.0000 |
| Others | | | | | | |
| Triethanolamine | 0.2000 | 0.1000 | 0.2000 | 0.2000 | 0.2000 | 0.2000 |
| Aquaflex XL-30*⁹ | 3.0000 | 3.0000 | 3.0000 | 3.0000 | 3.0000 | 3.0000 |
| Cosmedia ATH*⁴ | 1.0000 | 1.0000 | 1.0000 | 1.0000 | 1.0000 | 1.0000 |

*¹Methylparaben from Nipa
*²Water/Glycerin/PEG-100 Stearate/Benzyl Alcohol/Palmitoyl Pentapeptide-3 from Croda Inc.
*³PEG-4 Laurate/PEG-6 Dilaurate/Iodopropynyl Butylcarbamate/PEG-4 from Lonza Inc.
*⁴Sodium Polyacrylate/Ethylhexyl Stearate/Trideceth-6 from Cognis Corp.
*⁵Laureth-4 from Croda Inc.
*⁶Acrylates/C10-30 Alkyl Acrylate Crosspolymer from Lubrizol Advanced Inc.
*⁷Water/Glycerin/Decyl Glucoside/Benzyl Alcohol/Lactic Acid/Palmitoyl Dipeptide No. 7 from Croda Inc.
*⁸Panthenol from DSM Corp.
*⁹Polyimide-1/Water from ISP Inc.
*¹⁰Polyethylene from Equistar Chemical
*¹¹Undecylenoyl phenylalanine from SEPPIC
*¹²Dimethicone/Dimethiconol from Dow Corning Inc.
*¹³Pentylene Glycol from Symrise Inc.
*¹⁴Water/Peucedanum Graveolens (Dill) Extract/Phenoxyethanol/Xanthan Gum from BASF Inc.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin-care composition comprising:
a dill extract;
a safe and effective amount of a peptide;
a safe and effective amount of a hydrating skin active; and
a dermatologically acceptable carrier.

2. The skin-care composition of claim 1 wherein the peptide is selected from the group consisting of pentapeptides, dipeptides, and combinations thereof.

3. The skin-care composition of claim 2 wherein the pentapeptide is palmitoyl pentapeptide-3.

4. The skin-care composition of claim 2 wherein the dipeptide is selected from the group consisting of carnosine, palmitoyl dipeptide-7, and combinations thereof

5. The skin-care composition of claim 1 wherein the hydrating skin active is selected from the group consisting of vitamin B₃ compounds, caffeine, and combinations thereof.

6. The skin-care composition of claim 1 wherein the dermatologically acceptable carrier is in the form of an oil-in-water emulsion.

7. The skin-care composition of claim 1 wherein the dermatologically acceptable carrier is in the form of an aqueous gel.

8. The skin-care composition of claim 1 further comprising one or more optional skin-care actives that are selected from the group consisting of vitamins, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, antifungals, and combinations thereof.

9. The skin-care composition of claim 1 further comprising an optional skin-care active, wherein the optional skin-care active is N-Acetyl Glucosamine.

10. The skin-care composition of claim 1 further comprising an optional skin-care active, wherein the optional skin-care active is hexamidine isethionate.

11. A method of producing a chronic skin-firming benefit, said method comprising the step of:
a) topically applying to the skin of a mammal in need of such treatment, a safe and effective amount of a skin-care composition according to claim 1.

12. A skin-care composition comprising:
a dill extract;
a safe and effective amount of a peptide;
a safe and effective amount of a hydrating skin active;
a safe and effective amount of an acute skin-care active; and
a dermatologically acceptable carrier.

13. The skin-care composition of claim 12 wherein the acute skin-care active is selected from the group consisting of polyethylene, polyimide-1, and combinations thereof.

14. The skin-care composition of claim 12 wherein the peptide is selected from the group consisting of pentapeptides, dipeptides, and combinations thereof.

15. The skin-care composition of claim 14 wherein the pentapeptide is palmitoyl pentapeptide-3.

16. The skin-care composition of claim 14 wherein the dipeptide is selected from the group consisting of carnosine, palmitoyl dipeptide-7, and combinations thereof.

17. The skin-care composition of claim 12 wherein the hydrating skin active is selected from the group consisting of vitamin B₃ compounds, caffeine, and combinations thereof.

18. The skin-care composition of claim 12 wherein the dermatologically acceptable carrier is in the form of an oil-in-water emulsion.

19. The skin-care composition of claim 12 wherein the dermatologically acceptable carrier is in the form of an aqueous gel.

20. The skin-care composition of claim 12 further comprising one or more optional skin-care actives that are selected from the group consisting of vitamins, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, antifungals, and combinations thereof.

21. The skin-care composition of claim 12 further comprising an optional skin-care active, wherein the optional skin-care active is N-Acetyl Glucosamine.

22. The skin-care composition of claim 12 further comprising an optional skin-care active, wherein the optional skin-care active is hexamidine isethionate.

23. A method of producing a chronic skin-firming benefit and an acute skin-tightening sensation, said method comprising the step of:
a) topically applying to the skin of a mammal in need of such treatment, a safe and effective amount of a skin-care composition according to claim 12.

24. A skin-care composition comprising:
from about 0.001% to about 7.0% of a dill extract;
from about 1×10⁻⁷% to about 20% of a peptide selected from the group consisting of palmitoyl pentapeptide-3, carnosine, palmitoyl dipeptide-7, and combinations thereof;
from about 0.01% to about 50% of a hydrating skin active selected from the group consisting of vitamin B₃ compounds, caffeine, and combinations thereof;
from about 0.0001% to about 25% of an acute skin-care active selected from the group consisting of polyethylene, polyimide-1, and combinations thereof and a dermatologically acceptable carrier.
